SEQUENCE LISTING (36 sequences)

SEQ ID NO: 1 - protein, 9 aa, Artificial Sequence
  Description of Artificial SequenceSynthetic
CASKMKSAC

SEQ ID NO: 2 - protein, 9 aa, Artificial Sequence
  Description of Artificial SequenceSynthetic
CASKMRSAC

SEQ ID NO: 3 - protein, 9 aa, Artificial Sequence
  Description of Artificial SequenceSynthetic
CASKMRSVC

SEQ ID NO: 4 - protein, 9 aa, Artificial Sequence
  Description of Artificial SequenceSynthetic
CASKMRSLC

SEQ ID NO: 5 - protein, 9 aa, Artificial Sequence
  Description of Artificial SequenceSynthetic
CASKMRSIC

SEQ ID NO: 6 - protein, 9 aa, Artificial Sequence
  Description of Artificial SequenceSynthetic
CASKMRLIC

SEQ ID NO: 7 - protein, 9 aa, Artificial Sequence
  Description of Artificial SequenceSynthetic
CASKMKLIC

SEQ ID NO: 8 - protein, 9 aa, Artificial Sequence
  Description of Artificial SequenceSynthetic
CASKMRAIC

SEQ ID NO: 9 - protein, 9 aa, Artificial Sequence
  Description of Artificial SequenceSynthetic
CASRMKLIC

SEQ ID NO: 10 - protein, 9 aa, Artificial Sequence
  Description of Artificial SequenceSynthetic
CILKMRSVC

SEQ ID NO: 11 - protein, 9 aa, Artificial Sequence
  Description of Artificial SequenceSynthetic
CILKMRSLC

SEQ ID NO: 12 - protein, 9 aa, Artificial Sequence
  Description of Artificial SequenceSynthetic
CASICCLIC

SEQ ID NO: 13 - protein, 9 aa, Artificial Sequence
  Description of Artificial SequenceSynthetic
CILKMRSIC

SEQ ID NO: 14 - protein, 9 aa, Artificial Sequence
  Description of Artificial SequenceSynthetic
CILKMRLIC

SEQ ID NO: 15 - protein, 9 aa, Artificial Sequence
  Description of Artificial SequenceSynthetic
CILKMKLIC

SEQ ID NO: 16 - protein, 9 aa, Artificial Sequence
  Description of Artificial SequenceSynthetic
CILRARLIC

SEQ ID NO: 17 - protein, 9 aa, Artificial Sequence
  Description of Artificial SequenceSynthetic
CILKMRAIC

SEQ ID NO: 18 - protein, 9 aa, Artificial Sequence
  Description of Artificial SequenceSynthetic
CASKMKLLC

SEQ ID NO: 19 - protein, 9 aa, Artificial Sequence
  Description of Artificial SequenceSynthetic
CASKMRVLC

SEQ ID NO: 20 - protein, 9 aa, Artificial Sequence
  Description of Artificial SequenceSynthetic
CASKMRLVC

SEQ ID NO: 21 - protein, 9 aa, Artificial Sequence
  Description of Artificial SequenceSynthetic
CLLRMRSAC

SEQ ID NO: 22 - protein, 9 aa, Artificial Sequence
  Description of Artificial SequenceSynthetic
CILRMRSAC

SEQ ID NO: 23 - protein, 9 aa, Artificial Sequence
  Description of Artificial SeqeuenceSynthetic
CVLRMRSAC

SEQ ID NO: 24 - protein, 9 aa, Artificial Sequence
  Description of Artificial SequenceSynthetic
CLIRMRSAC

SEQ ID NO: 25 - protein, 9 aa, Artificial Sequence
  Description of Artificial SequenceSynthetic
CLVRMRSAC

SEQ ID NO: 26 - protein, 9 aa, Artificial Sequence
  Description of Artificial SequenceSynthetic
CLLKMRSAC

SEQ ID NO: 27 - protein, 9 aa, Artificial Sequence
  Description of Artificial SequenceSynthetic
CLLRMKSAC

SEQ ID NO: 28 - protein, 9 aa, Artificial Sequence
  Description of Artificial SequenceSynthetic
CLLRMRSLC

SEQ ID NO: 29 - protein, 9 aa, Artificial Sequence
  Description of Artificial SequenceSynthetic
CLLRMRSVC

SEQ ID NO: 30 - protein, 9 aa, Artificial Sequence
  Description of Artificial SequenceSynthetic
CLLRMRSIC

SEQ ID NO: 31 - protein, 9 aa, Artificial Sequence
  Description of Artificial SequenceSynthetic
CALRMRSIC

SEQ ID NO: 32 - protein, 9 aa, Artificial Sequence
  Description of Artificial SequenceSynthetic
CLARMRSIC

SEQ ID NO: 33 - protein, 9 aa, Artificial Sequence
  Description of Artificial SequenceSynthetic
CLLRARSIC

SEQ ID NO: 34 - protein, 9 aa, Artificial Sequence
  Description of Artificial SequenceSynthetic
CLLRMASIC

SEQ ID NO: 35 - protein, 9 aa, Artificial Sequence
  Description of Artificial SequenceSynthetic
CLLRMRAIC

SEQ ID NO: 36 - protein, 9 aa, Artificial Sequence
  Description of Artificial SequenceSynthetic
CILKMKSAC

(19) United States
(12) Patent Application Publication
(10) Pub. No.: US 2005/0119186 A1
(43) Pub. Date: Jun. 2, 2005

(54) Potent peptide inhibitors and methods of use

(76) Inventor: Richard S. Larson, Albuquerque, NM (US)

Correspondence Address: Henry D. Coleman;Coleman Sudol Sapone, P.C., 714 Colorado Avenue, Bridgeport, CT, 06605-1601, US

(21) Appl. No.: 10/895,564

(22) Filed: Jul. 21, 2004

Related U.S. Application Data
(60) Provisional application No. 60/488,813, filed on Jul. 22, 2003.

(51) Int. Cl. A61K 38/08; C07K 7/06

(52) U.S. Cl. 514/16; 530/329

(57) ABSTRACT

The present invention relates to peptide compounds which modulate the interaction of ICAM-1 and LFA-1, and in particular, function as inhibitors of the interaction of integrins, more particularly, LFA-1, and one or several distinct intercellular adhesion molecules (ICAMS), in particular ICAM-1, pharmaceutical compositions comprising effective amounts of these peptide compounds and methods for the treatment and/or prevention of related disease states and conditions which are mediated through ICAM-1/LFA-1 interactions, for example, the interaction of cellular adhesion molecules with integrins and/or the emigration of leukocytes from blood into tissue.

### RELATED APPLICATIONS

This application claims the benefit of priority of provisional application 60/488,813, filed Jul. 22, 2003, which is incorporated in its entirety herein.

### BACKGROUND OF THE INVENTION

LFA-1 (lymphocyte function associated antigen-1) is an integrin ap heterodimer (Carlos and Harlan, 1994; Springer, 1994; Larson and Springer, 1990; McEver, 1990; Picker and Butcher, 1992). Although three other integrins restricted in expression to leukocytes share the same β subunit and have homologous α subunits (Mac-1, p150,95, and alpha d), only LFA-1 is expressed on normal and leukemia T cells (Larson and Springer, 1990). LFA-1 binds ICAM-1 (intracellular adhesion molecule), and although LFA-1 is constitutively expressed on all leukocytes, LFA-1 binding to ICAM-1 requires cellular activation. Activation, in part, results in conformational changes in LFA-1 that affect its avidity for ICAM-1. In contrast, ICAM-1 is constitutively avid and expressed on a wide array of cell types including leukocytes, endothelium, stromal cells, and fibroblasts. In a model developed by the present inventor, a stromal cell derived soluble factor cooperates with LFA-1 on the surface of T lineage acute lymphoblastic leukemia (T-ALL) cells (Winter et al., 1998). The LFA-1 on T-ALL cells results in bone marrow (BM) stromal cell binding via ICAM-1 that leads to enhanced leukemia cell survival. Furthermore, LFA-1/ICAM-1 dependent interaction between circulating leukocytes and endothelial cells lining blood vessels promotes extravasation of leukocytes into tisuue as seen in rheumatoid arthritis, myocardial infarction, stroke, transplant rejection, stroke and other inflammatory and immune mediated diseases as well as leukemia cells into tissue as seen in the life-threatening therapeutic complication of acute leukemia, retinoic acid syndrome (Brown et al., 1999). Hence, the development of effective in vivo inhibitors of LFA-1/ICAM interaction would be useful in the therapy of immune and inflammatory mediated diseases as well as leukemia and its complications.

The present inventor has shown, for example, that inhibition of LFA-1/ICAM-1 dependent stromal cell binding with mAbs decreases survival of T-ALL cell lines and T-ALL cells isolated from patients. In one study, a representative sample from a patient with T-ALL showed that survival of T-ALL cells is augmented by BM stromal cells and that survival is inhibited by mAbs directed against LFA-1 (mAb TSI/22,5 μg/ml) or its ligand ICAM-1 (mAb 84H10, 10 μg/ml). This observation has been replicated for T-ALL cell lines Jurkat and Sup T I as well as a subset of patients with T-ALL. However, even though in vivo use of mAbs against LFA-1 or ICAM-1 blocks LFA-1 function in a number of disease models, unfortunately anaphylactic reactions and secondary physiologic effects have hampered this approach (McMuray, 1996; DeMeester: et al., 1996; Jackson et al., 1997; Cuthbertson et al., 1997; Gundel et al., 1992; Haming et al., 1993; Nakano et al, 1995).

Another means to interfere with protein—protein interactions is through the use of small peptide inhibitors. In fact, small peptide inhibitors to adhesion molecules structurally-related to LFA-1 have recently been approved for clinical use in coagulopathies (Ohman et al., 1995; Adgey et al., 1998; Leficovis and Topol, 1995). Short linear peptides (<30 amino acids) have also been described that prevent or interfere with integrin dependent firm adhesion using sequences derived from integrin or their ligands. In particular, these peptides have been derived from a number of integrin receptors: the β2 and β3 subunits of integrins, and the α.sub.iib subunit of ICAM-1, and VCAM-1 (Murayama et al., 1996; Jacobsson and Frykberg, 1996; Zhang and Plow, 1996; Budnik et al., 1996; Vanderslice et al, 1997; Suchiro et al., 1996; Endemann et al., 1996). However, the clinical applicability of these linear peptides is limited. The half maximal inhibitory concentration (IC.sub.50; concentration at which aggregation is inhibited 50%) for most of these peptides is 10.sup.-4 M with purified receptor-ligand pairs (univalent interactions) and they are ineffective at inhibiting multivalent interactions, during cell-cell adhesion. In addition, linear peptides have short serum half-lives because of proteolysis. Therefore, prohibitively high concentrations of peptide would have to be administered in a clinical setting and a biologic effect would not necessarily occur.

Longer peptides, ranging in length from 25-200 residues, have also been reported to block β1, β2, and β3 integrin dependent adhesion (Zhang and Plow, 1996; Budnik et al., 1996; Vanderslice et al, 1997; Suchiro et al., 1996; Endeman et al., 1996). In general, these peptide inhibitors may have higher affinities or slower off-rates than short peptides and, therefore, are better inhibitors. However, they are still susceptible to proteolysis.

Therefore, a need exists to develop novel and specific classes of pharmaceutical agents to inhibit the binding of LFA-1 and ICAM-1 and to be useful in the treatment of hematopoietic neoplastic diseases as well as other diseases that involve emigration of leukocytes from blood into tissue, such as myocardial infarction, radiation injury, asthma, rheumatoid arthritis, and lymphoma metastasis, among numerous other diseases states or conditions as discussed and described herein.

### SUMMARY OF THE INVENTION

The present invention relates to peptide compounds which modulate the interaction of ICAM-1 and LFA-1, and in particular, function as inhibitors of the interaction of integrins, more particularly, LFA-1, and one or several distinct intercellular adhesion molecules (ICAMS), in particular ICAM-1, pharmaceutical compositions comprising effective amounts of these peptide compounds and methods for the treatment and/or prevention of related disease states and conditions which are mediated through ICAM-1/LFA-1 interactions, for example, the interaction of cellular adhesion molecules with integrins and/or the emigration of leukocytes from blood into tissue.

Without being limited by way of theory, compounds according to the present invention in at least one aspect inhibit the ICAM-1/LFA-1 dependent homotypic aggregation of human lymphocytes and human lymphocyte adherence to ICAM-1, and modulate immune cell activation/proliferation, for example, as competitive inhibitors of intercellular ligand/receptor binding reactions involving ICAMs and leukointegrins.

Thus, in an additional aspect of the present invention, the present compounds and compositions may be used to treat diseases and conditions such as an inflammatory or immune cell-mediated diseases including arthritis, reactive arthritis, rheumatoid arthritis, osteoarthritis, diseases or conditions resulting from non-specific immune responses such as adult respiratory distress syndrome, shock, oxygen toxicity, septic shock, multiple organ injury syndrome secondary to septicemia, multiple organ injury syndrome secondary to trauma, ischemia-reperfusion injury, reperfusion injury of tissue due to cardiopulmonary bypass, myocardial infarction, acute glomerulonephritis, vasculitis, reactive arthritis, dermatosis with acute inflammatory components, stroke, thermal injury, hemodialysis, leukapheresis, ulcerative colitis, necrotizing enterocolitis and granulocyte transfusion associated syndrome, autoimmune diseases including Raynaud's syndrome, autoimmune thyroiditis, dermatitis, multiple sclerosis, rheumatoid arthritis and osteoarthritis, insulin-dependent diabetes mellitus, diabetic retinopathy, uveitis, inflammatory bowel disease including Crohn's disease and ulcerative colitis, and systemic lupus erythematosus, solid organ transplant rejection, hyperproliferative diseases such as psoriasis, hyperkeratosis, ichthyosis, keratoderma, lichen planus or warts, hematopoietic neoplasms, including Hodgkin's disease, non-Hodgkin's lymphoma, leukemias, including non-acute and acute leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia, stem cell leukemia and metastasis of these diseases. Compositions according to the present invention may be used in adjunct therapy in reducing the likelihood of retinoic acid syndrome in an acute promyelocytic leukemia (APL) patient being treated with retinoic acid and can also be used to fluidize or dissolve a thrombus in a patient in combination with a thrombolysis agent.

In addition, compounds according to the present invention may be useful for reducing the likelihood of an allograft rejection, for example, an organ transplant rejection, especially including a heart, lung(s), kidney (renal), liver, bone marrow and thyroid transplant. Compounds according to the present invention may be used for treating diabetes, especially type I or type II diabetes mellitus, myocardial infarction, asthma radiation injury, or as an adjunct to minimize toxicity with cytokine therapy in the treatment of cancers. In general these compounds may be employed in the treatment of those diseases currently treatable through steroid therapy.

Compounds according to the present invention are cyclic nonapeptides represented by the following chemical formula I:
CX¹Y¹Z¹X²Y²Z²X³C
or a multimer (e.g., dimer, trimer, tetramer, pentamer, etc.) thereof where C is a cysteinyl residue;
X¹ is alanine, leucine or isoleucine;
Y¹ is serine or leucine;
Z¹ is lysine, arginine or isoleucine, preferably lysine or arginine, more preferably lysine;
X² is methionine, alanine or cysteine, more preferably methionine;
Y² is arginine, lysine or cysteine, more preferably arginine or lysine;
Z² is serine, leucine or alanine; and
X³ is leucine, isoleucine or valine;
   or pharmaceutically acceptable salts thereof.

Preferred peptide compounds according to the present invention are represented by the following peptide sequences:

| | |
|---|---|
| CASKMKSAC; | (SEQ ID NO:1) |
| CASKMRSAC; | (SEQ ID NO:2) |
| CASKMRSVC; | (SEQ ID NO:3) |
| CASKMRSLC; | (SEQ ID NO:4) |
| CASKMRSIC; | (SEQ ID NO:5) |
| CASKMRLIC; | (SEQ ID NO:6) |
| CASKMKLIC; | (SEQ ID NO:7) |
| CASKMRAIC; | (SEQ ID NO:8) |
| CASRMKLIC; | (SEQ ID NO:9) |
| CILKMRSVC; | (SEQ ID NO:10) |
| CILKMRSLC; | (SEQ ID NO:11) |
| CASICCLIC; | (SEQ ID NO:12) |
| CILKMRSIC; | (SEQ ID NO:13) |
| CILKMRLIC; | (SEQ ID NO:14) |
| CILKMKLIC; | (SEQ ID NO:15) |
| CILRARLIC; | (SEQ ID NO:16) |
| CILKMRAIC; | (SEQ ID NO:17) |
| CASKMKLLC; | (SEQ ID NO:18) |
| CASKMRVLC; | (SEQ ID NO:19) |
| and | |
| CASKMRLVC. | (SEQ ID NO:20) |

Of the above compounds, the most preferred are CASKMRSAC (SEQ ID NO:2) and CILKMRSVL (SEQ ID NO:10).

The above peptide compounds can be formulated into pharmaceutical compositions which comprise an effective amount of at least one of the above-described peptide compounds optionally in combination with a pharmaceutically acceptable carrier, additive or excipient.

### DESCRIPTION OF THE FIGURES

FIG. 1 shows the accuracy of the QSAR equation (Eq. (3)) for the LFA-1/ICAM-1 training set. The points lie near a 45 line and the points in the test set are accurately predicted, indicating that the QSAR equation is a good correlation of signatures to the IC₅₀ values.

FIG. 2 shows the distribution of IC₅₀ values for the solutions of the inverse-QSAR using six signatures. Solutions are grouped according to their IC₅₀ values: 0-100, 101-200, 201-300, up to 1000.

FIG. 3 shows reconstruction of a solution peptide from the amino acid signature. Since the structure is cyclic, it does not matter which signature is used to start of the sequence. Here we choose a C(AC) to start. This is connected to both an A and a C. Selecting the signature A(CS) we know that it is already connected to a C, so the next signature must be S(AK). Continuing in this manner, the last signature should match up the first.

### DETAILED DESCRIPTION OF THE INVENTION

The following terms are used throughout the specification to describe the present invention. Where a term is not given a specific definition herein, the term is to be given the same meaning as understood by those of ordinary skill in the art. The definitions given to the disease states or conditions which may be treated using one or more of the compounds according to the present invention are those which are generally known in the art.

The term “patient” or “subject” is used throughout the specification to describe an animal, preferably a human, to whom treatment, including prophylactic treatment, with the compositions according to the present invention is provided. For treatment of those infections, conditions or disease states which are specific for a specific animal such as a human patient, the term patient refers to that specific animal.

The term “compound” is used herein to refer to any specific chemical compound disclosed herein. Within its use in context, the term generally refers to a single oligopeptide, but in certain instances may also refer to stereoisomers and/or optical isomers (including racemic mixtures) of disclosed compounds.

The term “effective amount” is used throughout the specification to describe concentrations or amounts of compounds according to the present invention which may be used to produce a favorable change in a disease or condition treated, whether that change is a remission, a favorable physiological result, a reversal or attenuation of a disease state or condition treated, the prevention or the reduction in the likelihood of a condition or disease-state occurring, depending upon the disease or condition treated. Where compounds are used in combination, each of the compounds is used in an effective amount, wherein an effective amount may include a synergistic amount.

The term “ICAM-1/LFA-1 mediated disease” is used throughout the specification to describe a disease which is mediated through the interaction of ICAM-1 with LFA-1, for example, by inhibiting the ICAM-1/LFA-1 dependent homotypic aggregation of human lymphocytes and human lymphocyte adherence to ICAM-1, or modulating immune cell activation/proliferation, for example, as competitive inhibitors of intercellular ligand/receptor binding reactions involving CAMS and leukointegrins. The present compounds and compositions may be used to treat varied diseases and conditions such as an inflammatory or immune cell-mediated diseases including arthritis, rheumatoid arthritis, osteoarthritis, diseases or conditions resulting from non-specific immune responses such as adult respiratory distress syndrome, shock, oxygen toxicity, septic shock, multiple organ injury syndrome secondary to septicemia, multiple organ injury syndrome secondary to trauma, ischemia-reperfusion injury, reperfusion injury of tissue due to cardiopulmonary bypass, myocardial infarction or use with thrombolysis agents to liquidize or eliminate thrombus, acute glomerulonephritis, vasculitis, reactive arthritis, dermatosis with acute inflammatory components, stroke, thermal injury, hemodialysis, leukapheresis, ulcerative colitis, necrotizing enterocolitis and granulocyte transfusion associated syndrome, solid organ transplant rejection, autoimmune diseases including Raynaud's syndrome, autoimmune thyroiditis, dermatitis, multiple sclerosis, arthritis, including rheumatoid arthritis and osteoarthritis, insulin-dependent diabetes mellitus, diabetes retinopathy, uveitis, inflammatory bowel disease including Crohn's disease and ulcerative colitis, and systemic lupus erythematosus, hyperproliferative diseases such as psoriasis, hyperkeratosis, ichthyosis, keratoderma, lichen planus or warts, hematopoietic neoplasms and metastasis of such neoplasms, including Hodgkin's disease, non-Hodgkin's lymphoma, leukemias, including non-acute and acute leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; and in adjunct therapy in reducing the likelihood of retinoic acid syndrome in an acute promyelocytic leukemia (APL) patient being treated with retinoic acid. The compounds according to the present invention may also be used to fluidize or dissolve a thrombus in a patient, preferably in combination with a thrombolysis agent.

The term “neoplasia” or “neoplasm” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and may invade surrounding tissues. As used herein, the term neoplasia/neoplasm is used to describe all cancerous disease states and embraces or encompasses the pathological process associated with cancer, in particular hematopoietic neoplasm and its metastasis. A hematopoietic neoplasm is a neoplasm of hematopoeitic cells of the blood or lymph system and includes disease states such as Hodgkin's disease, non-Hodgkin's lymphoma, leukemias, including non-acute and acute leukemias, such as acute myelogenous leukemia, acute lymphocytic leukemia, acute promyelocytic leukemia (APL), adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia.

The term “prophylactic” is used to describe the use of a compound described herein which either prevents or reduces the likelihood of a condition or disease state in a patient or subject.

The term “multimer” is used to describe peptide compounds according to the present invention which are used as multiples of the nine amino acid units found in the simplest peptide compounds according to the present invention. For example, a dimer is a peptide of 18 amino acid units (9 units in each of the two monomeric units forming the dimer), whereas a trimer is a peptide of 27 amino acid units (9 units in each of the three monomeric units forming the trimer). The individual units preferably may be linked be disulfide bonds between cysteinyl residues in each of the nonapeptides, or alternatively, may be linked by peptide bonds at the amino or carboxy terminus of the individual nonapeptide monomeric units. Multimeric compounds according to the present invention are preferably no more than dodecamers (12 monomeric units), are more preferably dimers or trimers, even more preferably dimers.

The term “pharmaceutically acceptable” refers to a salt form of the present compounds or a carrier, additive or excipient which is not unacceptably toxic to the subject to which it is administered.

The present disclosure provides novel peptide containing compositions that inhibit the interaction of integrins with ICAMS and in particular, LFA-1/ICAM-1 interaction. Preferred are compositions including nine amino acid peptides in which the terminal amino acids are cysteines, thus allowing the peptide to exist in a heterodetic cyclic form by disulfide bond formation of the mercaptide groups in the terminal cysteine amino acids or in a homodetic form by amide peptide bond formation between the terminal amino acids. Cyclizing small peptides through disulfide or amide bonds between the N- and C-terminus cysteines may circumvent problems of affinity and half-life. Disulfide bonds connecting the amino and carboxy terminus decrease proteolysis and also increase the rigidity of the structure, which may yield higher affinity compounds. Peptides cyclized by disulfide bonds have free amino- and carboxy-termini which still may be susceptible to proteolytic degradation, while peptides cyclized by formation of an amide bond between the N-terminal amine and C-terminal carboxyl, no longer contain free amino or carboxy termini.

Cyclic peptides may have longer half-lives in serum (see, for example, Picker and Butcher, Ann. Rev: Immunol., 1992; Huang et al., Biopolymers, 45, 367, 1997). Moreover, the side-effects from peptide therapy are minimal, since anaphylaxis and immune responses against the small peptide occur only rarely (Ohman et al., Eur. Heart J, 16, 50, 1995; Adgey, Amer. Heart J., 135, S43, 1998). Finally cyclic peptides have been shown to be effective inhibitors in vivo of integrins involved in human and animal disease (Jackson et al., J. Med. Chem., 40, 3359, 1997; Cuthbertson et al., J. Med. Chem., 40, 2876, 1997; Leficovis and Topol, Curr. Opin. Cardiology, 10, 420, 1995; Goligorsky et al., Gun. Exper. Pharm. Physiol., 25, 276,1998; Ojima et al., Bioorg. Med. Chem, 3, 337, 1995; and Noiri et al., Kidney Intl., 46, 1050, 1994). Thus, the peptides of the present invention, other than CASICCLIC (SEQ ID NO:12), can be linked either by a C—N linkage or a disulfide linkage.

The present invention is not limited in any way by the method of cyclization of peptides, but encompasses peptides whose cyclic structure may be achieved by any suitable method of synthesis. Thus, heterodetic linkages may include, but are not limited to formation via disulfide, alkylene or sulfide bridges. Methods of synthesis of cyclic homodetic peptides and cyclic heterodetic peptides, including disulfide, sulfide and alkylene bridges, are disclosed in U.S. Pat. No. 5,643,872, herein incorporated in entirety by reference. Other examples of cyclization methods are discussed and disclosed in U.S. Pat. No. 6,008,058, herein incorporated in entirety by reference. Cyclic peptides can also be prepared by incorporation of a type II′ β-turn dipeptide (Doyle et al., Int. J. Peptide Protein Res., 47, 427, 1996). In certain aspects, embodiments of the present invention include cyclic peptides comprising the heptapeptides represented by residues 2 through 8 of the exemplified cysteine-containing nonapeptides. Thus, embodiments of the present invention include cyclic peptides comprising the nonapeptide sequences which are set forth hereinabove. In addition, oligomeric peptides comprising peptides which contain at least two of the above nonapeptides as dimers (18 amino acid units), trimers (27 amino acid units), tetramers (36 amino acid units), pentamers (45 amino acid units), etc., linked either through disulfide bonds or amide (peptide) bonds as otherwise described herein, are also contemplated by the present invention.

Compounds of the invention may be prepared readily using general peptide synthetic methods which are well known in the art. Preferably, the peptides are prepared stepwise, either from the carboxyl terminus or from the amine terminus, depending upon the general chemistry utilized, by adding the appropriate amino acid, to the peptide appropriate terminus as it is synthesized. Peptide synthesis on immobilized substrates, to facilitate isolation of the final product, may be preferred. If desired, intermediates and products may be purified by chromatography and/or recrystallization. Starting materials, amino acid intermediates and reagents are either commercially available or may be prepared by one skilled in the art using methods described in the chemical literature.

The present invention includes the compositions comprising the pharmaceutically acceptable acid or base addition salts of compounds of the present invention. The acids which are used to prepare the pharmaceutically acceptable acid addition salts of the aforementioned base compounds useful in this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among others.

Pharmaceutically acceptable base addition salts may also be used to produce pharmaceutically acceptable salt forms of the compounds according to the present invention. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e, calcium and magnesium), ammonium or water-soluble amine addition salts such as N-methylglucamine-(meglumine), and the lower alkanolammonium and other base salts of pharmaceutically acceptable organic amines, among others.

The novel peptide molecules of formula I provided by the invention inhibit the ICAM-1/LFA-1 dependent homotypic aggregation of human lymphocytes and human lymphocyte adherence to ICAM-1. While not being limited by way of theory, it is believe that these compounds have therapeutic utility in the modulation of immune cell activation/proliferation, e.g., as competitive inhibitors of intercellular ligand/receptor binding reactions involving CAMs and Leukointegrins.

Regardless of the mechanism, the compounds of the present invention may be used to treat conditions or disease states in patients or subjects who suffer from those conditions or disease states or are at risk for those conditions or certain inflammatory conditions, including conditions resulting from a response of the non-specific immune system in a mammal (e.g., adult respiratory distress syndrome, shock, oxygen toxicity, multiple organ injury syndrome secondary to septicemia, multiple organ injury syndrome secondary to trauma, reperfusion injury of tissue due to cardiopulmonary bypass, myocardial infarction or use with thrombolysis agents, acute glomerulonephritis, vasculitis, reactive arthritis, dermatosis with acute inflammatory components, stroke, thermal injury, hemodialysis, leukapheresis, ulcerative colitis, necrotizing enterocolitis and granulocyte transfusion associated syndrome) and conditions resulting from a response of the specific immune system in a mammal (e.g., psoriasis, organ/tissue transplant rejection, graft vs. host reactions and autoimmune diseases including Raynaud's syndrome, autoimmune thyroiditis, dermatitis, multiple sclerosis, rheumatoid arthritis, insulin-dependent diabetes mellitus, uveitis, inflammatory bowel disease including Crohn's disease and ulcerative colitis, and systemic lupus erythematosus), hyperproliferative diseaes, hematopoietic neoplasms. The compounds of the invention may also be used in treating asthma or as an adjunct to minimize toxicity with cytokine therapy in the treatment of cancers. In general these compounds may be employed in the treatment of those diseases currently treatable through steroid therapy.

The compounds of the present invention may be used to treat hematopoietic neoplasms and their metastasis including, for example, Hodgkin's disease, non-Hodgkin's lymphoma, leukemias, including non-acute and acute leukemias, such as acute myelogenous leukemia, acute lymphocytic leukemia, acute promyelocytic leukemia (APL), acyte T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia. In addition, the present compounds may be useful in adjunct therapy in reducing the likelihood of retinoic acid syndrome in an acute promyelocytic leukemia (APL) patient being treated with retinoic acid.

Thus, another aspect of the invention is the provision of a method for the treatment or prophylaxis of the above-described conditions through the adminstration of therapeutic or prophylactic amounts of one or more compounds of formula I.

In accordance with the method provided by the invention, the novel compounds of formula I may be administered for either a “prophylactic” or “therapeutic” purpose either alone or with other agents, including other immunosuppressive or antiinflammatory agents or other anti-cancer agents. When provided prophylactically, the immunosuppressive compound(s) are provided in advance of any inflammatory response or symptom (for example, prior to, at, or shortly after the time of an organ or tissue transplant but in advance of any symptoms of organ rejection). The prophylactic administration of a compound of the formula I serves to prevent or attenuate any subsequent inflammatory response (such as, for example, rejection of a transplanted organ or tissue, etc.). The therapeutic administration of a compound of the formula I serves to attenuate any actual inflammation (such as, for example, the rejection of a transplanted organ or tissue). Thus, in accordance with the invention, a compound of the formula I can be administered either prior to the onset of inflammation (so as to suppress an anticipated inflammation) or after the initiation of inflammation.

The novel compounds of the formula I may, in accordance with the invention, be administered in single or divided doses by the oral, parenteral or topical routes. Administration of the active compound may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal and suppository administration, among other routes of administration. Enteric coated oral tablets may also be used to enhance bioavailability of the compounds from an oral route of administration. The most effective dosage form will depend upon the pharmacokinetics of the particular agent chosen as well as the severity of disease in the patient. Administration of compounds according to the present invention as sprays, mists, or aerosols for intra-nasal, intra-tracheal or pulmonary administration may also be used. The present invention therefore also is directed to pharmaceutical compositions comprising an effective amount of compound according to the present invention, optionally in combination with a pharmaceutically acceptable carrier, additive or excipient.

The amount used is that amount effective within the context of the administration. A suitable oral dosage for a compound of formula I would be in the range of about 0.01 mg to 10 g or more per day, preferably about 0.1 mg to about 1 g per day. In parenteral formulations, a suitable dosage unit may contain from 0.1 to 250 mg of said compounds, which may be administered from one to four times per day, whereas for topical administration, formulations containing 0.01 to 1% active ingredient are preferred. It should be understood, however, that the dosage administration from patient to patient will vary and the dosage for any particular patient will depend upon the clinician's judgment, who will use as criteria for fixing a proper dosage the size and condition of the patient as well as the patient's response to the drug.

When the compounds of the present invention are to be administered by the oral route, they may be administered as medicaments in the form of pharmaceutical preparations which contain them in association with a compatible pharmaceutical carrier material. Such carrier material can be an inert organic or inorganic carrier material suitable for oral administration. Examples of such carrier materials are water, gelatin, talc, starch, magnesium stearate, gum arabic, vegetable oils, polyalkylene-glycols, petroleum jelly and the like.

The pharmaceutical preparations can be prepared in a conventional manner and finished dosage forms can be solid dosage forms, for example, tablets, dragees, capsules, and the like, or liquid dosage forms, for example solutions, suspensions, emulsions and the like.

The pharmaceutical preparations may be subjected to conventional pharmaceutical operations such as sterilization. Further, the pharmaceutical preparations may contain conventional adjuvants such as preservatives, stabilizers, emulsifiers, flavor-improvers, wetting agents, buffers, salts for varying the osmotic pressure and the like. Solid carrier material which can be used include, for example, starch, lactose, mannitol, methyl cellulose, microcrystalline cellulose, talc, silica, dibasic calcium phosphate, and high molecular weight polymers (such as polyethylene glycol).

For parenteral use, a compound according to the present invention can be administered in an aqueous or non-aqueous solution, suspension or emulsion in a pharmaceutically acceptable oil or a mixture of liquids, which may contain bacteriostatic agents, antioxidants, preservatives, buffers or other solutes to render the solution isotonic with the blood, thickening agents, suspending agents or other pharmaceutically acceptable additives. Additives of this type include, for example, tartrate, citrate and acetate buffers, ethanol, propylene glycol, polyethylene glycol, complex formers (such as EDTA), antioxidants (such as sodium bisulfite, sodium metabisulfite, and ascorbic acid), high molecular weight polymers (such as liquid polyethylene oxides) for viscosity regulation and polyethylene derivatives of sorbitol anhydrides. Preservatives may also be added if necessary, such as benzoic acid, methyl or propyl paraben, benzalkonium chloride and other quaternary ammonium compounds.

The compounds of this invention may also be administered as solutions for nasal application and may contain in addition to the compounds of this invention suitable buffers, tonicity adjusters, microbial preservatives, antioxidants and viscosity-increasing agents in an aqueous vehicle. Examples of agents used to increase viscosity are polyvinyl alcohol, cellulose derivatives, polyvinylpyrrolidone, polysorbates or glycerin. Preservatives added may include benzalkonium chloride, chloro-butanol or phenylethyl alcohol, among numerous others.

Additionally, the compounds provided by the invention can be administered by suppository.

In certain aspects according to the present invention, where various cancers are to be treated, the compounds may be co-administered with at least one other anti-cancer agent such as antimetabolites, Ara C, etoposide, doxorubicin, taxol, hydroxyurea, vincristine, cytoxan (cyclophosphamide) or mitomycin C, among numerous others, including topoisomerase I and topoisomerase II inhibitors, such as adriamycin, topotecan, campothecin and irinotecan, other agent such as gemcitabine and agents based upon campothecin and cis-platin. By “co-administer” it is meant that the present compounds are administered to a patient such that the present compounds as well as the co-administered compound may be found in the patient's bloodstream at the same time, regardless when the compounds are actually administered, including simultaneously. In many instances, the co-administration of the present compounds with traditional anticancer agents produces a synergistic (i.e., more than additive) result which is unexpected.

The present invention also relates to pharmaceutical compositions comprising a compound according to the present invention in combination with a thrombolysis agent (such as streptokinase, tissue plasminogen activator, anisoylated plasminogen streptokinase activator complex or mixtures, thereof) to fluidize or dissolve a thrombus in a patient, optionally in combination with a pharmaceutically acceptable carrier, additive or excipient.

The following examples are included to demonstrate representative embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Identification of Inhibitory Peptides According to the Present Invention Using QSAR

Following the teachings of Churchwell, et al., Jour. Mol. Graph. Model., 22, 263-273 (2004) and J. Chem. Inf. Comput. Sci., 43, 707-720 (2003) as described therein, a number of inhibitory peptides according to the present invention were identified.

The inverse-QSAR approach, was applied to a small set of inhibitory peptides directed against leukocyte trafficking and localization whose synthesis and testing in clinical trials is limited. A crucial event in leukocyte localization is binding to endothelium and subsequent migration from the blood into tissue. Recently, identification of cell surface adhesion molecules that mediate the adhesion of leukocytes to the endothelium, such as leukocyte functional antigen-1 (LFA-1) and its ligand intercellular adhesion molecule-1 (ICAM-1), have allowed investigation into leukocyte trafficking (E. C. Butcher, L. J. Picker, Lymphocyte homing and homeostasis, Science 272 (1996) 60-66, T. A. Springer, Traffic signals for lymphocyte recirculation leukocyte emigration: the multistep paradigm, Cell 76 (1996) 314, T. M. Carlos, J. M. Harlan, Leukocyte-endothelial adhesion molecules, Blood 84 (1994) 2068-2101). A novel antagonist of ICAM-1 has been developed that has in vivo efficacy (J. Shannon, M. Silva, D. Brown, R. S. Larson, Novel cyclic peptide inhibits ICAM-1 mediated cell aggression, J. Pept. Res. 58 (2001) 40-150, J. Shannon, D. C. Brown, M. Silva, R. S. Larson, A cyclic peptide inhibits LFA-1/ICAM-1 mediated cell aggregation, J. Pept. Res. 58 (2001) 1-14, L. Sillerud, E. Burks, D. C. Brown, R. S. Larson, NMR-derived solution model of potent ICAM-1 inhibitory peptide, J. Pept. 62 (2003) 97-116, S. H. Merchant, D. M. Gurule, R. S. Larson, Amelioration of ischemia reperfusion injury with cyclic peptide blockade of ICAM-1, Am. Phys-Heart Circ. 284 (2003) H1260-H1268), and Kelly and coworkers (T. A. Kelly, D. D. Jeanfarve, D. W. McNeil, et al., Cutting edge: a molecule antagonist of LFA-1 mediated cell adhesion, J. Immunol. 163 (1999) 5173-5177, K. Last-Barney, W. Davidson, M. Cardozo, et al., Binding elucidation of hydantoin-based antagonists of LFA-1 against multidisciplinary technologies: evidence for the allosteric inhibition protein-protein interaction, J. Am. Chem. Soc. 123 (2001) 5643-5650) have developed a small molecule antagonist of LFA-1, although toxicity effects have limited its development as an in vivo inhibitor.

The inhibitory compound tested was a cyclic peptide containing nine amino acids that bind to ICAM-1, thereby inhibiting LFA-1/ICAM-1 binding. Using alanine replacement and homologous amino acid substitutions, we identified residues strategic to the antagonist activity were identified. A small set of such derived peptides was used as a training set for the inverse-QSAR. The activity or potency of the peptides is associated with an IC₅₀ value, which measures the concentration that leads to half-maximal inhibition of receptor to ligand. Table 1, below, lists the amino acid sequence of sixteen such derived peptides with their IC₅₀ values (given in μM), determined using a cellular aggregation blocking assay (J. Shannon, D. C. Brown, M. Silva, R. S. Larson, A cyclic peptide inhibits LFA-1/ICAM-1 mediated cell aggregation, J. Pept. Res. (2001) 1-14, L. Sillerud, E. Burks, D. C. Brown, R. S. Larson, NMR-derived solution model of potent ICAM-1 inhibitory peptide, J. Pept. 62 (2003) 97-116).

TABLE 1

Training and test set for the ICAM-1/LFA-1
inhibitory peptides

| | | |
|---|---|---|
| | Ex- | |
| | perimental | Inhibitor |
| Peptide sequence | IC₅₀ (μM)^{a} | strength |

| | | | | |
|---|---|---|---|---|
| 1 | CLLRMRSAC | (SEQ ID NO:21) | 480 | Strong |
| 2 | CLLRMRSAC | (SEQ ID NO:22) | 190 | Strong |
| 3 | CVLRMRSAC | (SEQ ID NO:23) | >1000 | Non |
| 4 | CLIRMRSAC* | (SEQ ID NO:24) | 720 | Weak |
| 5 | CLVRMRSAC | (SEQ ID NO:25) | >1000 | Non |
| 6 | CLLKMRSAC | (SEQ ID NO:26) | 105 | Strong |
| 7 | CLLRMKSAC | (SEQ ID NO:27) | 90 | Strong |
| 8 | CLLRMRSLC | (SEQ ID NO:28) | >1000 | Non |
| 9 | CLLRMRSVC | (SEQ ID NO:29) | 700 | Weak |
| 10 | CLLRMRSIC | (SEQ ID NO:30) | 580 | Weak |
| 11 | CALRMRSIC | (SEQ ID NO:31) | >1000 | Non |
| 12 | CLARMRSIC | (SEQ ID NO:32) | >1000 | Non |
| 13 | CLLRARSIC* | (SEQ ID NO:33) | >1000 | Non |
| 14 | CLLRMASIC | (SEQ ID NO:34) | >1000 | Weak |
| 15 | CLLRMRAIC | (SEQ ID NO:35) | 710 | Weak |
| 16 | CILKMKSAC | (SEQ ID NO:36) | 40 | Strong |

*Peptides indicated with an asterisk (peptides 4 (SEQ ID NO:24) and 13 (SEQ ID NO:25) indicate compounds in the test set. Listed are the amino acid sequences, the experimentally determined IC₅₀ value and the potency of the peptide in inhibiting the ICAM-1/LFA-1 complex.
^{a}IC₅₀ values were determined using cellular assay described in Sillerud et al. (L. Sillerud, E. Burks, D. C. Brown, R. S. Larson, NMR-derived solution model of potent ICAM-1 inhibitory peptide, J. Pept. 62 (2003) 97-116).

In brief, the aggregation of a cell line dependent on LFA-1 binding to ICAM-1 was used to measure the inhibitory capacity of each peptide. Inhibitory peptides and cells were seeded in flat-bottomed microtiter plates and allowed to aggregate. The number of aggregates and total number of free (single) cells were counted using inverted phase microscopy. The percent aggregation, P, was determined as
P=100(1−F/I) (1)
where F is the final number of free cells and I is the initial number of free cells. This percent aggregation was then used to calculate the percent inhibition, Pi as follows
Pᵢ=100(1−Pᵢₚ/P_{c}) (2)
where Pᵢₚ is the percent aggregation with inhibitory peptide and P_{c} is the percent aggregation in the control experiment. The IC₅₀ values were calculated from a line fit to the percent inhibition data as a function of inhibitory peptide concentration over the range from 10 μM to 1 mM. Each condition was performed in duplicate while each experiment was performed a minimum of three times.

The first and last amino acids in the sequence are connected to one another via a disulfide bridge, making the structures cyclic. In addition, the peptides are classified according to their inhibitory capabilities: peptides with IC₅₀ values less than or equal to 500 are considered strong inhibitors, peptides with IC₅₀ values between 500 and 1000 are considered weak inhibitors and peptides with IC₅₀ values greater than or equal to 1000 are said to be non-inhibitors.

Fourteen of these peptides were used as a training set for the inverse-QSAR process; peptides 2 and 33 were used as the test set. Here, the goal was to find any other compounds within the property space of the training set that similarly inhibit the binding of LFA-1/ICAM-1, but with greater efficacy, i.e. a lower IC₅₀ value.

3.1. QSAR Analysis

The training set contained 14 cyclic peptides having nine amino acids, which were expressed in terms of a linear, one letter amino acid sequence. Following the procedure previously outlined, 47 unique atomic signatures of height one were used, each of which was given an unknown occurrence number xᵢ (See Table 2).

TABLE 2
Height one amino acid signatures for the ICAM-1/LFA-1 training set

x₁ A(CL)
x₂ A(CS)
x₃ A(IR)
x₄ A(LR)
x₅ A(MS)
x₆ A(RR)
x₇ C(AC)
x₈ C(CI)
x₉ C(CL)
x₁₀ C(CV)
x₁₁ I(AC)
x₁₂ I(CL)
X₁₃ I(CS)
x₁₄ I(LR)
x₁₅ K(LM)
x₁₆ K(MS)
x₁₇ L(AC)
x₁₈ L(AR)
x₁₉ L(CI)
x₂₀ L(CL)
x₂₁ L(CS)
x₂₂ L(CV)
x₂₃ L(IK)
x₂₄ L(IR)
x₂₅ L(KL)
x₂₆ L(LR)
x₂₇ L(RV)
x₂₈ M(AR)
x₂₉ M(KK)
x₃₀ M(KR)
x₃₁ M(RR)
X₃₂ R(AL)
x₃₃ R(AM)
x₃₄ R(AS)
x₃₅ R(IM)
x₃₆ R(LM)
x₃₇ R(MS)
x₃₈ R(MV)
x₃₉ S(AI)
x₄₀ S(AK)
x₄₁ S(AR)
x₄₂ S(IR)
x₄₃ S(LR)
x₄₄ S(RV)
x₄₅ V(CL)
x₄₆ V(CS)
x₄₇ V(LR)

Once the compounds in the training set were expressed in terms of their signatures, a linear QSAR equation could be created. First, a 47×14 descriptor matrix was constructed, then screened for perfectly correlated rows, or rows containing identical entries. Recall that equivalent variables distort the multiple linear regression results if included in the analysis. In addition, rows containing a single or double entry were discarded in an attempt to generalize the signatures in the QSAR so that none would map to a specific activity. Second, the actual IC₅₀ value for non-binding peptides (IC₅₀>1000) was not experimental measured, thus we assigned them a value of 1000. Furthermore, to make all the values of the dependent variables the same order of magnitude, we used the base ten logarithm of each IC₅₀ value in the QSAR. Last, a forward stepping algorithm was applied to select the most statistically significant signatures, one at a time. A QSAR equation was chosen with six variables, where the variables xi are the occurrence numbers of the signatures listed in Table 2 above.
log₁₀(IC₅₀)=2.81−0.739x₂−0.574x₈+0.662x₁₃+0.728x₃₁+0.727x₄₁−0.644x₃₇ (3)

The training set contained biased activities; almost half of the compounds had activities equal to 1000, the other majority of compounds contained activities less than 500. This trend was inevitably captured in the QSAR equations, where the added signatures simply distinguished between strong and non-inhibitory compounds. Thus, the coefficients in the QSAR equation are not as stable as we would like; ideally, they should exhibit little to no variation when another descriptor is added. However, since our data set is small, the QSAR will be sensitive to perturbations, i.e. the addition of new signatures.

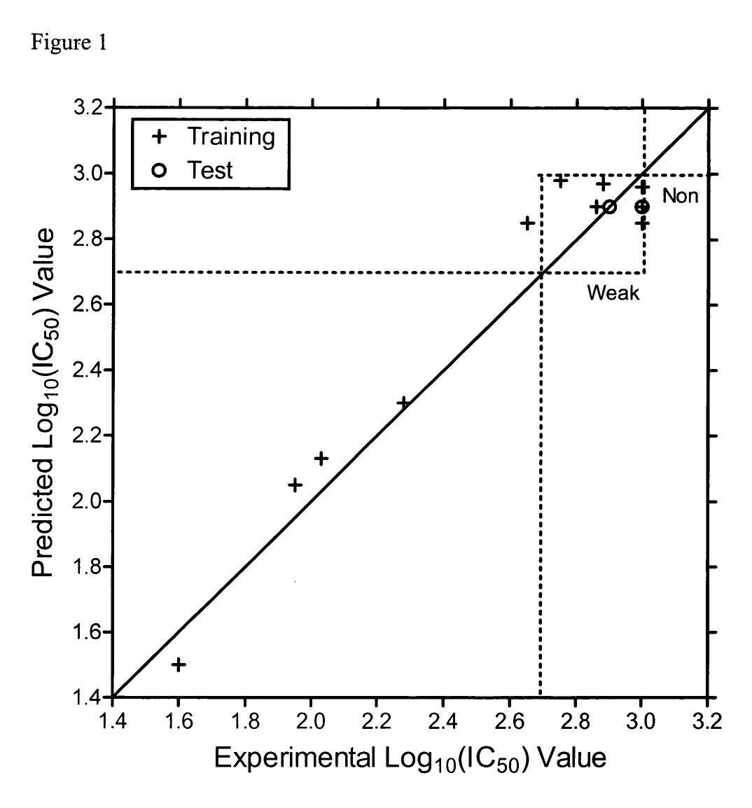

FIG. 1 illustrates the ability of Eq. (3) to correlate the IC₅₀ values of the training set as well as predict the values of the peptides in the test set. Although the statistics in Table 3 could be higher, the key is to choose a QSAR equation that not only correlates the signatures to the activities, but one that is also predictive. The QSAR chosen was based on the statistics in Table 4 (which show our QSAR has not been overly affected by multicolinearity) as well the QSAR's ability to predict the IC₅₀ values for compounds in the test set. Table 5 lists the differences of the predicted and experimental IC₅₀ values for the compounds in the test set using Eq. (3).

TABLE 3
Overall statistics for the QSAR equation with six signatures

| F | R² | s² | s² (test set) |
|---|---|---|---|
| 16.9 | 0.935 | 0.015 | 0.011 |

TABLE 4
Individual descriptor statistics for the QSAR equation with six signatures

| Descriptor | R² | Variable inflation factor | P-value |
|---|---|---|---|
| ^{x}2 | 0.3735 | 1.5962 | 0.0202 |
| ^{x}8 | 9.55e−7 | 1.00000095 | 0.9974 |
| ^{x}13 | 0.1692 | 1.2037 | 0.1439 |
| ^{x}31 | 0.4726 | 1.8961 | 0.0066 |
| ^{x}37 | 0.1609 | 1.1918 | 0.1551 |
| ^{x}41 | 0.0057 | 1.0057 | 0.7976 |

TABLE 5
The predicted IC₅₀ values and their differences for peptides in the
test set using Eq. (3).

| | | Experimental | Predicted | |
|---|---|---|---|---|
| | Peptide | IC₅₀ (μm) | IC₅₀ (μm) | Difference |
| 4 | CLIRMRSAC | 720 | 727.8 | 7.8 |
| 13 | CLLRARSIC | >1000 | 790.7 | 209.3 |

Construction of Constraint Equations

The amino acids can be regarded as vertices of degree 2. Consequently, the graphicality equation will always be satisfied and need not be calculated for this particular training set.

The consistency equations were calculated from the unique signature set as described above. In addition, it was desired that the resulting compounds be cyclic structures composed of nine amino acids. To capture this requirement, we added a constraint that the number of amino acids in any solution was to total 9. These equations are listed in Table 6. Notice that the individual constraint equations do not contain the majority of the variables. The two modulus equations (Table 6, Eqs. (16) and (23)) were incorporated into the system of equations by adding dummy variables (one for each modulus equation) to make them homogeneous.

TABLE 6
Constraint equations for the height one amino acid signatures in the
training set

| | |
|---|---|
| (1) | −x44 + x46 = 0 |
| (2) | −x38 + x47 = 0 |
| (3) | −x22 − x27 + x45 + x47 = 0 |
| (4) | −x10 + x45 + x46 = 0 |
| (5) | −x34 − x37 + x41 + x42 + x43 + x44 = 0 |
| (6) | −x21 + x43 = 0 |
| (7) | −x16 + x40 = 0 |
| (8) | −x13 + x39 + x42 = 0 |
| (9) | −x2 − x5 + x39 + x40 + x41 = 0 |
| (10) | −x28 − x30 − 2x31 + x33 + x35 + x36 + x37 + x38 = 0 |
| (11) | −x18 − x24 − x26 − x27 + x32 + x36 = 0 |
| (12) | x14 + x35 = 0 |
| (13) | −x3 − x4 − 2x6 + x32 + x33 + x34 = 0 |
| (14) | −x15 − x16 + 2x29 + x30 = 0 |
| (15) | −x5 + x28 = 0 |
| (16) | (x20 + x25 + x26)%2 = 0 |
| (17) | −x15 + x23 + x25 = 0 |
| (18) | −x12 − x14 + x19 + x23 + x24 = 0 |
| (19) | −x9 + x17 + x19 + x20 + x21 + x22 = 0 |
| (20) | −x1 − x4 + x17 + x18 = 0 |
| (21) | −x8 + x11 + x12 + x13 = 0 |
| (22) | −x₃ + x11 = 0 |
| (23) | (x7 + x8 + x9 + x10)%2 = 0 |
| (24) | −x1 − x2 + x7 = 0 |

Eqs. (16) and (23) are modulus equations, which can be expressed as homogeneous equations by adding a dummy variable. For example Eq. (16) would read x20 + x25 + x26 − 2z1 = 0. The % sign indicates the modulus is to be used.

Equation Solver

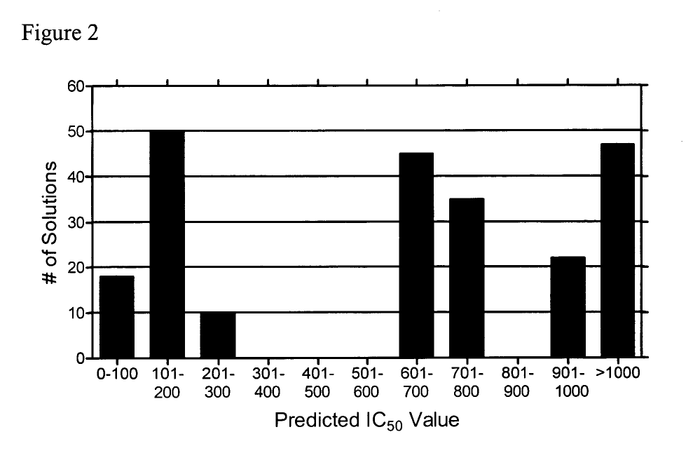

As mentioned previously, the inhomogeneous equations were intentionally excluded from the system in order to obtain results in a reasonable amount of time. Thus, only the constraint equations were solved using the Diophantine solver. Due to the size constraint of the peptides, only those solutions containing nine or less amino acids were kept, the rest were discarded. Solutions with less than nine amino acids were used in making linear combinations, again, adhering to the size constraint of nine amino acids. By leaving out the QSAR equation, all solutions were obtained with activities spanning a wide range of IC₅₀ values. The distribution of predicted activities is given in FIG. 2, where the solutions were divided into bins of 100 ranging up to 1000.

Structure Generator

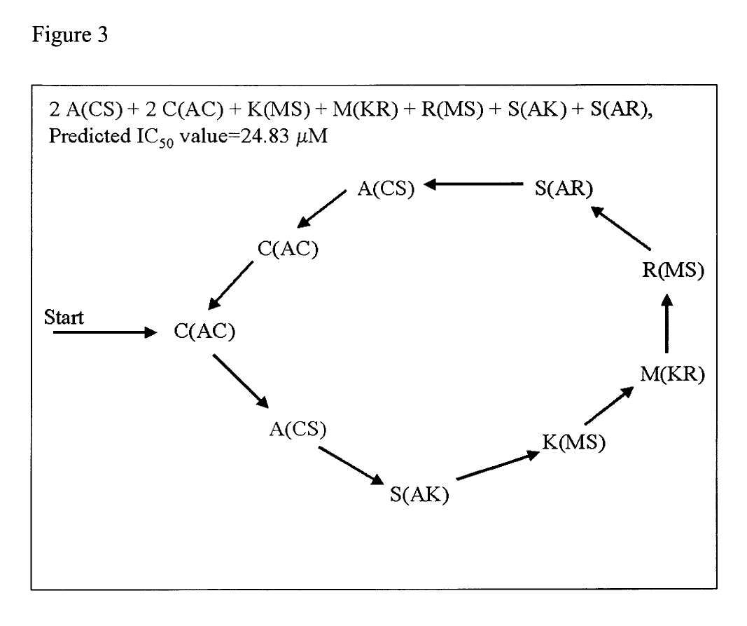

The reconstruction of the peptides was straightforward in this case. By construction, each peptide only contained nine amino acids that formed a cyclic structure. Therefore, once the amino acid sequence of the peptide was known, the structure would also be known. From a solution, we start building the amino acid sequence by selecting a descriptor—it does not matter which one since the structure is cyclic. The children of each amino acid are used as guides to tell us what the previous and following amino acids are in the sequence. FIG. 3 illustrates how a sample solution is reconstructed from the amino acid signatures. Here we pick a signature, in this case C(AC), since we know that the first and last amino acids form a disulfide linkage. We know that it is connected to another signature with root A and a signature with root C, both of which should have C as their child. So, we choose the signature A(CS) as the next residue in the sequence. C is already connected to an A, so the next residue must be a signature with root S. This process is reiterated until no more amino acids are left and the last amino acid should be a child of the first one and vice versa.

Table 7 lists 20 sequences corresponding to compounds with the lowest predicted IC50 values. Even though some of the peptides are predicted to be strong inhibitors, they may not be viable candidates for synthesis. For example peptide 12, which has the sequence CASICCLIC, contains two cysteine residues in the middle of the compound. These residues contain sulfur atoms which may form undesired disulfide bonds that potentially distort the three dimensional structure.

TABLE 7

Peptide sequences of the twenty lowest IC₅₀
values as predicted by the inverse-QSAR with
six signatures

| | | |
|---|---|---|
| | Predicted | |
| | IC₅₀ value | Actual IC₅₀ |
| Peptide sequence | (μm) | value (μm) |

| | | | | |
|---|---|---|---|---|
| 1 | CASKMKSAC | (SEQ ID NO:1) | 21.48 | |
| 2 | CASKMRSAC | (SEQ ID NO:2) | 24.83 | 23 |
| 3 | CASKMRSVC | (SEQ ID NO:3) | 25.53 | |
| 4 | CASKMRSLC | (SEQ ID NO:4) | 25.53 | |
| 5 | CASKMRSIC | (SEQ ID NO:5) | 31.26 | |
| 6 | CASKMRLIC | (SEQ ID NO:6) | 31.41 | |
| 7 | CASKMKLIC | (SEQ ID NO:7) | 31.41 | |
| 8 | CASKMRAIC | (SEQ ID NO:8) | 31.41 | |
| 9 | CASRMKLIC | (SEQ ID NO:9) | 36.31 | |
| 10 | CILKMRSVC | (SEQ ID NO:10) | 37.33 | 28 |
| 11 | CILKMRSLC | (SEQ ID NO:11) | 37.33 | |
| 12 | CASICCLIC | (SEQ ID NO:12) | 38.46 | |
| 13 | CILKMRSIC | (SEQ ID NO:13) | 45.71 | |
| 14 | CILKMRLIC | (SEQ ID NO:14) | 45.92 | |
| 15 | CILKMKLIC | (SEQ ID NO:15) | 45.92 | |
| 16 | CILRARLIC | (SEQ ID NO:16) | 45.92 | |
| 17 | CILKMRAIC | (SEQ ID NO:17) | 45.92 | |
| 18 | CASKMKLLC | (SEQ ID NO:18) | 117.8 | |
| 19 | CASKMRVLC | (SEQ ID NO:19) | 117.8 | |
| 20 | CASKMRLVC | (SEQ ID NO:20) | 117.8 | |

Discussion of QSAR Results

From the inversion process, a total of 223 compounds were found, including the 14 original compounds in the training set and the two test set compounds. The trends found in the training set reappear in FIG. 2. Recall that activities in the training set are biased towards either the strong or non-inhibitory groups. A similar trend emerges in the predicted activities of the solution set, with a gap in IC₅₀ values ranging between 300 and 600. This can be controlled with larger training set.

The goal of the inverse-QSAR method was to predict, if any, novel inhibitory compounds processing a lower IC₅₀ value than those in the training set. There were 77 new peptides classified as strong inhibitors. Of these, 12 represent peptides with predicted IC₅₀ values less than 40—the IC₅₀ value of peptide 16, which was the strongest inhibitor in the training set. To provide feedback on these predictions, two of these peptides were synthesized, sequences 2 and 10, using cellular assays. Their experimental IC₅₀ values were very close to the predicted values (see Table 7), and appear to be the strongest inhibiting peptides that work in-vivo as well.

### Description of Biological Properties and Examples

The biological properties of representative compounds of formula I are investigated by way of the experimental protocol described below. This protocol and related experiments are also described in U.S. Pat. Nos. 6,353,013 and 6,630,447, relevant portions of which are incorporated by reference herein. Compounds according to the present invention which exhibit inhibitory activity in the described assay are potential candidate compounds for treatment of disease states and conditions otherwise described herein.

Assay to Determine Inhibition of LFA-1 Binding to ICAM-1

Purpose of Assay:

The described assay protocol is designed to study the direct antagonism, by a test compound, of the interaction of the CAM, ICAM-1 with the Leukointegrin CD 18/CD11a (LFA-1).

Assay Protocol:

LFA-1 is immunopurified using the TS2/4 antibody from a 20 g pellet of human JY or SKW3 cells, utilizing a protocol previously described (Dustin, M. J.; et al., J. Immunol. 1992, 148, 2654-2660). The LFA-1 is purified from SKW3 lysates by immunoaffinity chromatography on TS2/4 LFA-1 mAb Sepharose and eluted at pH 11.5 in the presence of 2 mM MgCl₂ and 1% octylglucoside. After collection and neutralization of fractions from the TS2/4 column, samples are pooled and precleared with Protein G agarose.

A soluble form of ICAM-1 is constructed, expressed, purified and characterized as previously described (Marlin, S.; et al., Nature, 1990, 344, 70-72 and see Arruda, A.; et al., Antimicrob. Agents Chemother. 1992, 36, 1186-1192). Briefly, isoleucine 454 which is located at the putative boundary between domain 5 of the ectodomain and the transmembrane domain, is changed to a stop codon using standard oligonucleotide-directed mutagenesis. This construction yields a molecule identical with the first 453 amino acids of membrane bound ICAM-1. An expression vector is created with a hamster dihydrofolate reductase gene, a neomycin-resistance marker, and the coding region of the sICAM-1 construct described above, along with the promoter, splice signals, and polyadenylation signal of the SV40 early region. The recombinant plasmid is transfected into CHO DUX cells using standard calcium phosphate methods. Cells are passaged in selective media (G418) and colonies secreting sICAM-1 are amplified using methotrexate. sICAM-1 is purified from serum-free media using traditional non-affinity chromatographic techniques, including ion exchange and size exclusion chromatography.

LFA-1 binding to ICAM-1 is monitored by first incubating sICAM-1 at 40 μg/mL in Dulbecco's phosphate buffered saline with calcium and magnesium, additional 2 mM MgCl.sub.2 and 0.1 mM PMSF (Diluting Buffer) in a 96-well plate for 30 min at room temperature. Plates are then blocked by the addition of 2% (w/v) bovine serum albumin in Diluting Buffer for 37.degree. C. for 1 h. Blocking solution is removed from wells, and test compounds are diluted and then added followed by the addition of approximately 25 ng of immunoaffinity purified LFA-1. The LFA-1 is incubated in the presence of test compound and ICAM-1 at 37° C. for 1 h. Wells are washed 3 times with Diluting Buffer. The bound LFA-1 is detected by the addition of a polyclonal antibody directed against a peptide corresponding to the CD 18 cytoplasmic tail in a 1:100 dilution with Diluting Buffer and 1% BSA and allowed to incubate for 45 min at 37° C. Wells are washed 3 times with Diluting Buffer and the bound polyclonal antibody is detected by the addition of a 1:4000 dilution of horse radish peroxidase conjugated to goat immunoglobulin directed against rabbit immunoglobulin. This reagent is allowed to incubate for 20 min at 37° C., wells are washed as above and the substrate for the horse radish peroxidase is added to each well to develop a quantitative colorimetric signal proportional to the amount of LFA-1 bound to sICAM-1. Soluble ICAM-1 (60 μg/mL) is used as a positive control for inhibition of the LFA-1/ICAM-1 interaction. The lack of the addition of LFA-1 to the binding assay is used as a background control for all samples. A dose-response curve may be generated for all test compounds. Those compounds exhibiting a K_{d} of less than 10 μM are viewed as being effective inhibitors and having potential use as therapeutic agents in the present invention.

Note that the in vitro test results obtained from the above experiments described above correspond to in vivo activity as shown by Merchant, et al., Am. J. Physiol Heart Circ. Physiol., 284: H1260-1268, 2003.

### ALTERNATIVE EXAMPLES

Phage Display and Consensus Sequence

As a part of the present disclosure, phage display is used to identify peptide sequences that bind ICAM-1 and block LFA-1/ICAM interaction. Briefly a library of cysteine-constrained heptapeptides is purchased from New England Labs (Cambridge, Mass.) and screened for its ability to bind the LFA-1 ligand, ICAM-1. Human ICAM-1 has been previously isolated in functional form (Larson et al., Leukocyte Typing, p. 566, 1990, Larson, et al., Cell Regul., 1:359, 1990), and a variation of this technique is used to obtain purified recombinant soluble ICAM-1 for use in phage display. Each phage in the library has the potential to display a unique cyclic heptapeptide fused to its gene III coat on its surface. The linkage of the displayed random peptide with a phage surface protein forms the basis of the technique. The library consists of approximately 2.8×10¹¹ random heptapeptide sequences expressed on phage, compared to 207 (20 possible amino acids in 7 different positions) or 1.28×10⁹ possible heptapeptide sequences. The phage are then screened for their ability to bind purified ICAM-1 by interaction with the displayed heptapeptide sequences. The phage are then screened for their ability to bind purified ICAM-1 by panning. Bound phage is eluted using the anti-ICAM-1 mAb 84h410. This mAb binds to amino acid residues on ICAM-1 that are similar to those to which LFA-1 binds (Staunton et al., Cell, 61:243, 1990). Elution with R6.5 allows for isolation of phage expressing cyclic peptides that bind a region on ICAM-1 that is shared with LFA-1 binding. In addition, phage are eluted with mAb for 1 hour so that the peptides with highest affinity and slower off-rates (i.e., peptides most likely to be potent in vivo inhibitors) would be included. Thus, peptide sequences that block ICAM-1/LFA-1 interactions are identified.

Adherent phage are selected and amplified in ER2537 bacteria through four rounds of panning. The sequences of 12-18 phage in each round are determined. A working consensus peptide is determined after nucleotide sequencing of 18 phage in the fourth and final round. The recurring amino acids form the basis of derivative structures. The ability of each phage isolated after four rounds of panning to specifically bind ICAM-1 is also determined in an ELISA assay with serial dilutions of phage.

Rapid Aggregation Assay for Screening Peptide Effectiveness

LFA-1 dependent cell aggregation has been previously studied using an aggregation assay it with a variety of leukocyte subclasses and cell lines (Larson et al., Leukocyte Typing, p. 566, 1990; Wang et al., J. Virol., 62, 4173, 1988; and Larson et al., Blood, 90, 2747, 1997). JY cells may be obtained form American Type Tissue Culture Collection and are maintained in RPMI 1640 supplemented with 10% FBS at 37° C. in 5% CO₂. Aggregation of cells are measured in a homotypic aggregation assay using ICAM-1 as a stimuli. JY cells are washed twice with serum free medium and resuspended at a concentration of 4×10⁵ is cells/ml. Cells are preincubated with the desired concentration of peptide for 15 min at room temperature. In a final volume of 100 μl, 50.mu.l of cells and peptide are seeded in 96 well flat bottomed microtiter plates. Cells are allowed to aggregate at 37° C. in humidified air with 5% CO₂. Cells are visualized and counted by inverted phase contrast microscopy at the time indicated. Within each well of aggregates as well as the total number of free (single) cells are counted. Percent aggregation was determined by the following equation:
Percent aggregation=100×(1−number of free cells/total number of cells)

Assay for Measuring LFA-1 Dependent Ex Vivo Survival of Leukemic Cells.

A co-culture assay has been developed by the present inventor that quantifies ex vivo survival of T-ALL cells (Winter et al., Blood, 89 (suppl. 1):8 la, 1998). Using this assay, survival of T-ALL cell lines as well as T-ALL cells isolated from patients requires LFA-1 binding to ICAM-1 on bone marrow (BM) derived stromal cells. In this assay cryopreserved or fresh leukemic samples are seeded onto HS5 stromal cell monolayers in 24 well plates. The stromal cell line HS5 has been previously shown to support complete hematopoiesis of normal precursor cells (Roecklein and Torak-Storb, Blood, 85:97, 1995). HS5 cells are λ-irradiated with 2500 cGy, a dose that has been determined to prevent stromal cell proliferation over 168 hours. Leukemia cells are harvested at 1 and 96 hours. The number of leukemic cells recovered is measured in a flow cytometer by techniques based on those known in the art (Manabe et al., Blood, 79, 2370, 1992; and Manabe et al., Blood, 83, 758, 1994). The leukemia cells are stained by direct immunofluorescence using a fluoroisothiocyanate (FITC) labeled mAb directed against a pan T-ALL antigen CD5 as described (Larson et al., Leukocyte Typing, p. 566, 1990; Larson and McCurley, Am J Clin Path, 104, 204, 1995). A gate is set around the area of light scatter where the viable CD5 positive T-ALL cells are found at the beginning of the cultures. Then, the T-ALL cells with predetermined light scattering and CD5 presentation are enumerated by counting the number of events passing through the gate in a 60 second time period. In each analysis 5×10⁵ fluorescent Immuno-Chek beads (Coulter, Hialeah, Fla.) are added to each sample. The number of beads that pass through the flow cytometer in 60 seconds is also counted, allowing the measured bead number to serve as an internal control for the volume that passes through the flow cytometer in 60 seconds. The calculation is as follows: (number of CD5 T-ALL cells/volume passed through flow cytometer as determined by fluorescent beads) X volume of the sample=the number of cells in the sample. The percentage of survival is calculated by: (number of cells in test sample at t-96 h/number of cell in sample at t-1 h)×100.

Parallel Plate Flow Chamber for Measuring Leukocyte-Endothelial Cell Interaction under Physiologic Flow Conditions In Vivo.

An assay has been developed that provides an in vitro model of neutrophils or APL cells binding to activated endothelium. The binding of APL cells using a parallel plate flow chamber recapitulates events that occur in retinoic acid syndrome (Larson et al., Blood, 90, 2747, 1997; Brown et al., Brit. J Haematol., 107, 86, 1999). A parallel plate flow chamber simulates the physiologic flow conditions in blood and adhesive interactions in post-capillary venules. Post-capillary venules are the physiologically relevant locations of leukemia cell-endothelial cell interaction and extravasation. Since parallel plate flow chamber experiments have been shown to accurately recapitulate in vivo observations, a parallel plate flow chamber is used to examine the inhibitory effects of peptides on APL cell line binding and transmigration through endothelium under physiologic flow conditions. Monolayers of endothelial cells are placed in the parallel plate flow chamber, and the leukemic cells are pumped through the chamber at physiologic flow rates. The interaction between the flowing leukemia cells and the endothelium are videotaped microscopically, and the number of rolling, firmly adhered and transmigrated leukemia cells is quantified by computer-assisted image analysis.

With all-trans retinoic acid (ATRA) treatment, the APL cell line NB-4 acquires the ability to firmly attach to activated endothelium via LFA-1/ICAM-1 interaction. Inhibition of LFA-1/ICAM-1 interaction prevents firm adherence to and transmigration through endothelium of the APL cell line under physiologic flow. This has been demonstrated with monoclonal antibodies against LFA-1 and ICAM-1, which prevent firm attachment to and transmigration through activated endothelium of APL cells in a parallel plate flow chamber (Larson et al., 1997, supra; Brown et al, 1999, supra). Flowing ATRA-treated APL cell lines over activated endothelial cell monolayers in a parallel flow chamber determines the effectiveness of peptides to inhibit LFA-1 dependent firm adherence and subsequent transmigration under physiologic flow conditions. ICAM-1 expressed on activated endothelial monolayers are incubated with cyclic peptides over a range of concentrations (10⁻⁴ to 10⁻⁸ M) and the IC₅₀ is determined.

For investigating neutrophil binding, neutrophils are isolated from heparin anticoagulated venous blood of healthy adult donors by centrifugation on Ficoll-Hypaque density gradients as described by Simon et al., J. Immunol., 149, 2765, (1992). Isolated neutrophils are suspended at a concentration of 10⁷/ml in Hanks' Balanced Salt Solution supplemented with 10 mmol/L HEPES, pH 7.4 and 0.2% human serum albumin (Armour Pharmaceutical, Kankakee, Ill.) and used within 2 hours of preparation. Neutrophils are kept on ice and resuspended in RPMi pre-warmed to 37° C. immediately before use.

Monoclonal Antibodies

In order to have adequate monoclonal antibodies at a reasonable cost, hybridomas were grown present inventor and mAbs were purified for blocking studies. The following monoclonal antibodies have been isolated from hybridoma supernatants: mAbs against LFA-1 (TS2/4 and TSI/22) and ICAM-1 (RR1/1, R6.5 and 84H10) (Larson et al, Blood, 90, 2747, 1997).

Screening Assay

The ability of candidate compounds to bind to ICAM-1 is assessed by a competitive binding assay. Candidate compounds may be peptide or non-peptide compounds. Binding to ICAM-1 is quantified by the ability to displace a peptide of the present invention, including the peptides of the present invention. The displaced peptide can be assayed by a number of techniques. For example, radiolabeled peptide can be synthesized using commercial available radiolabeled amino acids precursors. Peptides radiolabelled with H³, C¹⁴ or S³⁵ can be quantified by routine liquid scintillation techniques. Alternatively, a fluorescent labeled peptide can be synthesized. For example, lysine can be inserted in a non-critical position and labeled with fluroescein isothiocyanate (“FITC”). In addition to FITC, the peptide may be labeled with any suitable flurophore. A carboxy fluroescein derivative of one or more of the peptides of the present invention may be prepared. Alternatively, peptides cyclized with an amide peptide linkage have free sulfhydryl groups available for linkage to fluorescent compounds such as thiocyanates. Separation of bound from unbound peptide and quantitation of displaced peptide can be performed by routine techniques known to one of skill in the art. This embodiment of the invention is not limited by the method used to quantify the displaced peptide, and any suitable analytical technique may be used and be within the scope of the invention.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

1. A compound according to the formula:
CX¹Y¹Z¹X²Y²Z²X³C or a multimer thereof
where C is a cysteinyl residue;
X¹ is alanine or isoleucine;
Y¹ is serine or leucine;
Z¹ is lysine, arginine or isoleucine;
X² is methionine, alanine or cysteine;
Y² is arginine, lysine or cysteine;
Z² is serine, leucine or alanine; and
X³ is leucine, isoleucine or valine;
or pharmaceutically acceptable salts thereof.

2. A peptide according to claim 1 wherein
X¹ is alanine or isoleucine;
Y¹ is serine or leucine;
Z¹ is lysine or arginine;
X² is methionine;
Y² is arginine or lysine;
Z² is serine or leucine; and
X³ is isoleucine or valine;

3. A peptide according to claim 1 wherein
X¹ is alanine;
Y¹ is serine;
Z¹ is lysine or arginine;
X² is methionine;
Y² is arginine or lysine;
Z² is serine or leucine; and
X³ is leucine, isoleucine or valine.

4. A peptide according to claim 1 wherein
Z¹ is lysine;
X² is methionine;
Y² is arginine;
Z² is serine; and
X³ is isoleucine.

5. A peptide according to claim 1 which is a dimer, trimer, tetramer or pentamer.

6. A peptide according to claim 1 which is a dimer or trimer.

7. A peptide according to the formula:

| | |
|---|---|
| CASKMKSAC; | (SEQ ID NO:1) |
| CASKMRSAC; | (SEQ ID NO:2) |
| CASKMRSVC; | (SEQ ID NO:3) |
| CASKMRSLC; | (SEQ ID NO:4) |
| CASKMRSIC; | (SEQ ID NO:5) |
| CASKMRLIC; | (SEQ ID NO:6) |
| CASKMKLIC; | (SEQ ID NO:7) |
| CASKMRAIC; | (SEQ ID NO:8) |
| CASRMKLIC; | (SEQ ID NO:9) |
| CILKMRSVC; | (SEQ ID NO:10) |
| CILKMRSLC; | (SEQ ID NO:11) |
| CASICCLIC; | (SEQ ID NO:12) |
| CILKMRSIC; | (SEQ ID NO:13) |
| CILKMRLIC; | (SEQ ID NO:14) |
| CILKMKLIC; | (SEQ ID NO:15) |
| CILRARLIC; | (SEQ ID NO:16) |
| CILKMRAIC; | (SEQ ID NO:17) |
| CASKMKLLC; | (SEQ ID NO:18) |
| CASKMRVLC; | (SEQ ID NO:19) |
| and | |
| CASKMRLVC. | (SEQ ID NO:20) |

8. The peptide according to claim 7 according to the formula:

| | |
|---|---|
| CASKMRSAC | (SEQ ID NO:2) |
| and | |
| CILKMRSVL. | (SEQ ID NO:10) |

9. The peptide according to claim 8 which is

| | |
|---|---|
| CASKMRSAC. | (SEQ ID NO:2) |

10. The peptide according to claim 8 which is

| | |
|---|---|
| CILKMRSVL. | (SEQ ID NO:10) |

11. A pharmaceutical composition comprising an effective amount of a compound according to claim 1, optionally in combination with a pharmaceutically acceptable carrier, additive or excipient.

12. A method for treating an ICAM-1/LFA-1 mediated disease or condition comprising administering an amount of the compound according to any of claims 1-10 to a subject in need thereof effective to treat said ICAM-1/LFA-1 mediated disease or condition.

13. The method according to claim 12 wherein said disease is an inflammatory or immune cell-mediated disease, a disease or condition resulting from a non-specific immune response, an autoimmune disease, a hyperproliferative disease or condition or a hematopoietic neoplasm or its metastasis.

14. The method according to claim 13 wherein said disease is an inflammatory or immune cell-mediated disease.

15. The method according to claim 13 wherein said disease is arthritis, osteoarthritis, adult respiratory distress syndrome, psoriasis, shock, oxygen toxicity, septic shock, multiple organ injury syndrome secondary to septicemia, multiple organ injury syndrome secondary to trauma, ischemia-reperfusion injury, reperfusion injury of tissue due to cardiopulmonary bypass, myocardial infarction, acute glomerulonephritis, vasculitis, reactive arthritis, dermatosis with acute inflammatory components, stroke, thermal injury, asthma, solid organ transplant rejection, hemodialysis, leukapheresis, ulcerative colitis, necrotizing enterocolitis and granulocyte transfusion associated syndrome, Raynaud's syndrome, autoimmune thyroiditis, dermatitis, multiple sclerosis, rheumatoid arthritis, insulin-dependent diabetes mellitus, diabetes retinopathy, uveitis, inflammatory bowel disease, Crohn's disease, ulcerative colitis, systemic lupus erythematosus, Hodgkin's disease, non-Hodgkin's lymphoma and non-acute and acute leukemias.

16. The method according to claim 12 wherein said disease or condition is myocardial infarction, asthma, septic shock, diabetic retinopathy or solid-organ transplant rejection.

17. The method according to claim 16 wherein said disease or condition is myocardial infarction.

18. The method according to claim 16 wherein said disease or condition is asthma.

19. The method according to claim 16 wherein said disease or condition is septic shock.

20. The method according to claim 12 wherein said disease or condition is acute T-cell lymphoblastic leukemia, lymphoma or lymphoma metastasis.

21. The method according to claim 13 wherein said disease or condition is psoriasis, hyperkeratosis, ichthyosis, keratoderma, lichen planus or warts.

22. The method according to claim 21 wherein said disease or condition is psoriasis.

23. The method according to claim 13 wherein said hematopoietic neoplasm or its metastasis is Hodgkin's disease, non-Hodgkins lymphoma, acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia.

24. A method of fluidizing or dissolving a thrombus in a patient comprising administering a compound according to any of claims 1-10, optionally in combination with a thrombolysis agent in a pharmaceutical carrier, additive or excipient.

25. A method of reducing the likelihood of retinoic acid syndrome in an acute promyelocytic leukemia (APL) patient being treated with retinoic acid, said method comprising administering a compound according to any of claims 1-10 to said patient.

26. A pharmaceutical composition comprising a compound according to claim 1 in combination with a thrombolysis agent, optionally in combination with a pharmaceutically acceptable additive, carrier or excipient.

27. The composition according to claim 26 wherein said thrombolysis agent is streptokinase, tissue plasminogen activator, anisoylated plasminogen streptokinase activator complex or mixtures, thereof.

28. A pharmaceutical composition comprising a compound according to claim 1 in combination with an agent selected from the groups consisting of antimetabolites, Ara C, etoposide, doxorubicin, taxol, hydroxyurea, vincristine, cytoxan, mitomycin C, topoisomerase I and topoisomerase II inhibitors, gemcitabine and agents based upon campothecin and cis-platin.

29. A method of treating a hematopoietic neoplasm or its metastasis in a patient comprising administering a composition according to claim 27 to said patient.
